(19) United States
(12) Patent Application Publication
(10) Pub. No.: US 2010/0217058 A1
(43) Pub. Date: Aug. 26, 2010

(54) Drying Process for Linear Alpha Olefins

(76) Inventors: Peter Fritz, Unterhaching (DE); Heinz Bölt, Wolfratshausen (DE); Fuad Mosa, Riyadh (SA); Talal Ali, Riyadh (SA)

Correspondence Address: KRAMER LEVIN NAFTALIS & FRANKEL LLP;INTELLECTUAL PROPERTY DEPARTMENT, 1177 AVENUE OF THE AMERICAS, NEW YORK, NY, 10036, US

(21) Appl. No.: 11/990,163

(22) PCT Filed: Jun. 13, 2006
(86) PCT No.: PCT/EP2006/005643

§ 371 Date: Apr. 19, 2010

(30) Foreign Application Priority Data
Aug. 10, 2005 (EP) 05017340.0

(51) Int. Cl. C07C 2/08

(52) U.S. Cl. 585/520

(57) ABSTRACT

The present invention relates to a method for preparing linear alpha-olefins by oligomerisation of ethylene, comprising the steps of:
(i) oligomerising ethylene in a reactor in the presence of a solvent and a catalyst;
(ii) transferring a liquid organic outlet stream of the reactor, containing solvent, catalyst, dissolved ethylene and linear alpha-olefins, to a catalyst deactivation section;
(iii) deactivating the catalyst by washing the outlet stream with an aqueous basic phase to obtain a deactivated catalyst containing aqueous phase and a water saturated organic phase;
(iv) separating the aqueous phase and the organic phase from step (iii);
(v) transferring the water saturated organic phase to a distillation column;
(vi) distilling the water saturated organic phase; and
(vii) separating the distilled organic and aqueous phases.

The present invention relates to a method for preparing linear alpha-olefins (LAO).

Processes for the oligomerisation of ethylene utilizing a homogeneous catalyst are widely known. For example DE 43 38 414 C1 discloses a process for the oligomerisation of ethylene to obtain linear alpha-olefins, wherein ethylene is catalytically converted in an empty tubular reactor utilizing a catalyst comprising a zirconium component and an aluminum component. The process is advantageously carried out in a continuous mode wherein gaseous and liquid outlet streams are obtained. The liquid outlet stream usually contains solvent, catalyst, dissolved ethylene and linear alpha-olefins. To avoid plugging and reactor fouling the catalyst contained in the liquid outlet stream has to be immediately deactivated to avoid further oligomerisation.

According to the prior art, the obtained liquid outlet stream may be treated with water, alcohol or fatty acids to deactivate the catalytic components. Upon deactivation with water, large quantities of liquid linear alpha-olefins saturated with water are obtained, requiring the removal of the water for further processing. So far, water was removed by passing the liquid outlet stream through an adsorber, comprising for example molecular sieve, alumina or silica, to adsorb water and catalytic components thereon.

However, due to the large quantities of liquid linear alpha-olefins saturated with water obtained, excessively large adsorber vessels and thus high investment costs are required. Further, for regeneration of such large adsorbers, huge amounts of regeneration gas is required which also would result in high costs for the disposal thereof.

DE 198 07 226 A1 discloses a process for deactivation of complex metal organic catalysts in homogeneous processes, such as the oligomerisation of ethylene, wherein the obtained product solution is mixed with metal hydroxide in a protonic solvent, wherein the activation and isolation of the catalysts from the organic phase are carried out in one step. In this process the amount of the aqueous phase is kept relatively low, just enough to ensure the deactivation of the catalyst. This small amount of aqueous phase would be dissolved or entrained in the organic phase, but is too small for the formation of a separate aqueous phase.

It is therefore an object of the present invention to provide a method for preparing linear alpha-olefins which overcomes the drawbacks of the prior art, especially to provide a method comprising an improved drying step of the linear alpha-olefins obtained resulting in reduced costs, and reduced plant wastes.

This object is achieved by a method for preparing linear alpha-olefins by oligomerisation of ethylene, comprising the steps of:
(i) oligomerising ethylene in a reactor in the presence of a solvent and a catalyst;
(ii) transferring a liquid organic outlet stream of the reactor, containing solvent, catalyst, dissolved ethylene and linear alpha-olefins, to a catalyst deactivation section;
(iii) deactivating the catalyst by washing the outlet stream with an aqueous basic phase to obtain a deactivated catalyst containing aqueous phase and a water saturated organic phase;
(iv) separating the aqueous phase and the organic phase from step (iii);
(v) transferring the water saturated organic phase to a distillation column;
(vi) distilling the water saturated organic phase; and
(vii) separating the distilled organic and aqueous phases.

In a preferred embodiment the separation in step (iv) and/or (vii) takes place in a phase separator.

Also, after step (vii) the organic phase may be passed through an adsorber.

In addition, it is also preferred that the adsorber is zeolite, molecular sieve, alumina, silica, or mixtures thereof.

Preferably, the aqueous basic phase contains alkali metal hydroxide, preferably NaOH and/or KOH, NH₃, organic amines or mixtures thereof.

Moreover, after deactivating the catalyst with an aqueous basic phase in step (ii) the obtained organic phase may be additionally washed with water.

In one aspect the aqueous phases obtained in step (iv) and/or (vii) are recycled.

Surprisingly, it was found that utilizing the inventive method, only small or no adsorbers at all are required for removing the residual water contained in the organic stream of linear alpha-olefins. This is achieved by distilling the water saturated organic phase to remove the major amounts of water. After distillation, the organic phase may then, if necessary at all, passed through an adsorber. As only small or no adsorbers are required, reduced investments costs result. Further, only small amounts of regeneration gas are needed to regenerate the adsorber. Further, the inventive method results in a minimization of plant wastes, for example molecular sieve of the adsorber, regeneration offgas. Finally, the amount of consumed process water is reduced, since the water may be recovered for re-use. In the inventive method, larger quantities of aqueous phase are employed which allow a phase separation of an organic and an aqueous phase since this large quantities of aqueous phase cannot be dissolved in the organic phase. It is within the skill of an artisan to adjust the amount of aqueous solvent to obtain a phase separation.

Additional features and advantages of the invention method will now become apparent from the detailed description of the preferred embodiment thereof.

Ethylene is oligomerised in a suitable reactor, for example an empty tubular reactor, as disclosed in DE 43 38 414 C1, utilizing a catalyst comprising a zirconium component and an aluminum component. A suitable zirconium component is zirconium tetraisobutyrate, and a suitable aluminum component is ethyl aluminum sesquichloride.

The oligomerisation is carried out under conditions well known in the art. From the reactor, a liquid organic outlet stream is obtained, containing solvent (for example toluene), catalyst, ethylene dissolved in the solvent, and linear alpha-olefins. This liquid organic outlet stream is transferred to a catalyst deactivation section. To avoid plugging and fouling of the reactor and the pipes connecting the reactor and the catalyst deactivation section, it is preferred to deactivate the catalyst after exit from the reactor as soon as possible. According to the present invention, the catalyst is deactivated by washing the obtained organic outlet stream with an aqueous basic phase, for example water and sodium hydroxide. The organic phase and the aqueous basic phase may be separated, and, preferably, the obtained organic phase may be additionally washed with water, followed by a separation of the washed organic phase and the water.

The water saturated organic phase is then transferred to a distillation column. The distillation column may be designed to easily allow separation of the water contained in that organic phase. Especially, attention has to be drawn to avoid potential foaming and formation of hydrates. Foaming in distillation columns may be avoided by selection of appropriate operating conditions (pressure, temperature) and proper design of tray type and column geometry. Further, the formation of hydrates may be avoided by proper selection of the process conditions (e.g. pressure, temperature, tube wall temperature in heat exchangers, refrigerant temperature). However, the respective process design is well known for someone skilled in the art. After distillation, the water content of the bottoms product of the distillation column is significantly reduced due to the higher volatility of water compared to higher linear alpha-olefins forming substantially the bottoms product.

Most of the water contained in the water saturated organic phase is routed to the column overhead of the distillation column and may be condensed in its condenser together with light linear alpha-olefins. Thus, the water may be recovered in liquid state and may be separated from light linear alpha-olefins in a phase separator.

Since most of the water is recovered in the liquid phase, the residual water content in the linear alpha-olefins is significantly reduced.

To achieve required moisture levels of the final LAO products, if necessary, the organic phases may be passed through adsorbers. However, these adsorbers may be small compared to the prior art and thus reduce investments costs are necessary and also reduced amounts of regeneration gas to regenerate the adsorbers.

The features disclosed in the foregoing description and in the claims may, both separately and in any combination thereof, be material for realizing the invention in diverse forms thereof.

1. A method for preparing linear alpha-olefins by oligomerization of ethylene, comprising the steps of:
(i) oligomerising ethylene to linear alpha-olefins in a reactor in the presence of an organic solvent and a catalyst;
(ii) transferring a liquid organic outlet stream of the reactor, containing solvent, catalyst, dissolved ethylene and linear alpha-olefins, to a catalyst deactivation section;
(iii) deactivating the catalyst by washing the outlet stream with a basic aqueous solution;
(iv) separating the washed outlet stream from step (iii) into an aqueous phase comprised of deactivated catalyst and a water saturated organic phase comprised of organic solvent, linear alpha-olefin and dissolved ethylene;
(v) transferring the water saturated organic phase to a distillation column;
(vi) distilling the water saturated organic phase to yield a distillation overhead product containing most of the water in the water saturated organic phase and a distillation bottoms product containing most of the organics; and
(vii) separating the distillation overhead product to recover additional water.

2. A method according to claim 1, wherein the separation in step (iv) and/or (vii) takes place in a phase separator.

3. A method according to claim 1, wherein after step (vi) the distillation bottoms product is passed through an adsorber.

4. A method according to claim 3, wherein the adsorber comprised of zeolite, molecular sieve, alumina, silica, or mixtures thereof.

5. A method according to claim 1, wherein the basic aqueous solution comprises alkali metal hydroxide, NH₃, organic amines or mixtures thereof.

6. A method according to claim 1, wherein the water saturated organic phase is additionally washed with water.

7. A method according to claim 1, wherein the aqueous phase obtained in step (iv) and/or the water obtained in step (vii) are recycled.

8. The method according to claim 2, wherein after step (vi) the distillation bottoms product is passed through an adsorber comprised of zeolite, molecular sieve, alumina, silica, or mixtures thereof.

9. The method according to claim 5, wherein the basic aqueous solution comprises NaOH or KOH.

10. The method according to claim 4, wherein the basic aqueous solution comprises alkali metal hydroxide, NH₃, organic amines or mixtures thereof.

11. The method according to claim 10, wherein the water saturated organic phase is additionally washed with water.

12. The method according to claim 10, wherein the aqueous phase obtained in step (iv) and/or the water obtained in step (vii) are recycled.
